(19) United States
(12) Patent Application Publication
(10) Pub. No.: US 2018/0235568 A1
(43) Pub. Date: Aug. 23, 2018

(54) TRANSCRANIAL DOPPLER-BASED METHOD AND SYSTEM FOR DISPLAYING THREE-DIMENSIONAL INTRACRANIAL BLOOD FLOW INFORMATION

(71) Applicant: SHENZHEN DELICA MEDICAL EQUIPMENT CO., LTD., Shenzhen (CN)

(72) Inventors: Yi YANG, Shenzhen (CN); Yongping ZHENG, Shenzhen (CN); Chunhua LUO, Shenzhen (CN); Xiaoyi WANG, Shenzhen (CN)

(21) Appl. No.: 15/752,159

(22) PCT Filed: Nov. 12, 2015
(86) PCT No.: PCT/CN2015/094483

§ 371 Date: Feb. 12, 2018

(30) Foreign Application Priority Data
Oct. 29, 2015 (CN) 201510714916.8

(51) Int. Cl. A61B 8/06; A61B 8/08

(52) U.S. Cl. CPC A61B 8/06; A61B 8/466; A61B 8/483; A61B 8/488

(57) ABSTRACT

A transcranial Doppler-based method for displaying 3D intracranial blood flow information and a system thereof, comprising: A. performing multi-beam ultrasound scanning on predetermined intracranial areas using transcranial Doppler ultrasound probe, receiving ultrasonic echo signals; B. calculating, on basis of ultrasonic echo signal, to obtain blood flow information; C. obtaining blood flow information in a plurality of directions on basis of scanning of predetermined areas, forming stereoscopic models including blood flow information; D. visualizing data of stereoscopic model to form 3D images, and performing individualized adjustments to 3D images according to user commands, and outputting images and presenting to user. Compared with MRI, CT, DSA and the like, present invention is low-cost, convenient and capable of repeated detection. Present invention can significantly reduce degree of reliance on an operator's experience, provide blood flow information more complete and objective.

### CROSS-REFERENCES TO RELATED APPLICATIONS

This application is a national stage application of PCT Patent Application No. PCT/CN 2015/094483, filed on Nov. 12, 2015, which claims priority to Chinese Patent Application No. 201510714916.8, filed on Oct. 29, 2015, the content of all of which is incorporated herein by reference.

### FIELD OF THE INVENTION

The present invention relates to a field of medical ultrasound technology, and more particularly, to a transcranial Doppler-based method and system for displaying three-dimensional intracranial blood flow information.

### BACKGROUND

An ultrasonic transcranial Doppler blood flow analyzer is applied to detecting a spectrum of a blood flow in an intracranial arterial, and obtaining a plurality of information including a velocity and a direction of the blood flow, which has been applied commonly to a clinic practice. An intracranial test using the transcranial Doppler (TCD) commonly uses a plurality of following probe frequencies: a pulsed wave in 1.0 MHz, 1.6 MHz or 2.0 MHz.

Comparing to a color Doppler ultrasound, the TCD probes own a relatively low frequency and a relatively strong penetrating power, with a spectral image relatively rough and a resolution relatively poor. Currently, the TCDs are mainly applied to detecting blood flow information of a certain segment of an intracranial artery, or they might be able to give the blood flow information on the direction of an ultrasound beam, without being able to give a blood flow distribution figure in 2D or 3D.

While a technology of the color ultrasonic Doppler develops rapidly in recent years. A 3D imaging technology has been more and more mature and has been widely applied in a plurality of human body parts without any high-speed moving tissues, including a plurality of human abdominal organs, a fetus, a thyroid gland and more, using a plurality of 3D probes in a convex array or a linear array of the color ultrasonic Doppler (a mechanical sector scanning technology or a planar array technology).

However, these 3D imaging technologies are mainly focusing on a 3D structure of a plurality of extracranial organs and tissues, which may also perform a 3D reconstruction of blood flow in color by a Doppler imaging technology, but not an intracranial blood flow, and a price of such a product is much higher than that of a ultrasonic transcranial Doppler blood flow analyzer. Although the probes in a low frequency phased array of the color ultrasonic Doppler may be applied to an intracranial vascular detection, and being able to provide a 2D planar structure graph and a color blood flow display, or even a Doppler spectrum, it can not display a 3D blood vessels (or blood flow) Imaging.

A plurality of high-end imaging devices using other technologies being able to achieve a 3D display of the intracranial blood flow includes: MRI, CT, DSA and more, they may achieve the image information with a pretty high resolution. A 3D imaging technology of the color ultrasonic Doppler currently may not be able to achieve a plurality of blood flow parameters it displays at a same time. Due to a skull attenuates an ultrasonic signal, it makes the ultrasound in a mid or high frequency being able to achieve a clear image hard to penetrate the skull, while a probe in a low frequency ultrasounddoes not have a good imaging ability, thus currently in the market, there is no 3D probe and a related technology being able to be applied to intracranial blood vessels and tissues detection.

Also, some examinations using the MRI, CT and DSA, require a cooperation of a contrast agent, which owns certain radiation damage to a human body. A plurality of disadvantages of all these detection devices include that, they are costly and inconvenient to make a plurality of repeated examinations, not being able to be applied to a long-term monitoring, while owning a poor reproducibility, but achieving an imaging data of the blood flow at a separate time point only. Since there is no real information on a speed, direction and energy of the blood flow, there is no way to make a quantitative judgment on hemodynamics.

Therefore, the current technology needs to be improved and developed.

### BRIEF SUMMARY OF THE DISCLOSURE

According to the above described defects, the purpose of the present invention is providing a transcranial Doppler-based method and system for displaying three-dimensional intracranial blood flow information, aiming to solve the problem in the prior art that, there is no way to provide complete hemodynamic information while achieving the 3D distribution on the intracranial blood flow, thus achieving a long term continuous monitoring.

In order to achieve the above mentioned goals, the technical solution of the present invention to solve the technical problems is as follows:

a transcranial Doppler-based method for displaying three-dimensional intracranial blood flow information, wherein, the method includes:

A. using an ultrasonic probe of the transcranial Doppler to scan a plurality of predetermined intracranial areas with a multi-beam ultrasound, and receiving a plurality of ultrasonic echo signals;

B. according to the ultrasonic echo signals, calculating and achieving a plurality of blood flow information including a depth of a plurality of intracranial blood vessels, a blood flow direction, a relative blood flow volume and a blood flow velocity;

C. achieving the blood flow information in a plurality of directions based on the scanning of the predetermined areas, and forming a stereoscopic model containing the blood flow information based on a plurality of 3D modeling calculations;

D. forming a plurality of 3D images after visualizing the data of the stereoscopic model; and according to a user commands, personalize and adjust the 3D images, and outputting the image in order to present to the user.

The method for displaying three-dimensional intracranial blood flow information, wherein, the multi-beam ultrasound for scanning the predetermined intracranial areas is obtained by using a mechanical control method of a deflection of the ultrasonic probe or using an electron-focusing method of an ultrasonic multi-array element probe, or using a method of combining the mechanical control method and the electron-focusing method.

The method for displaying three-dimensional intracranial blood flow information, wherein, the method further includes: E. slice the stereoscopic model to calculate a diameter of the blood vessel; and calculate blood flow volume information of the blood vessel according to the diameter of the blood vessel and the speed of the blood flow.

The method for displaying three-dimensional intracranial blood flow information, wherein, the method further includes:

according to the 3D images, make a long term monitoring on an intracranial blood flow status in a whole, or on the blood flow information of one or a plurality of areas having a plurality of predetermined features.

The method for displaying three-dimensional intracranial blood flow information, wherein, the step B further includes: before running a 3D modeling calculation, filter out a plurality of non-blood flow information in the ultrasonic echo signals; and

according to a predetermined standard, adopt a calculation method with a different calculation complexity and accuracy for an ultrasonic echo signal in a different depth.

The method for displaying three-dimensional intracranial blood flow information, wherein, the method further includes: output the blood flow information to a sound card, and display to the user in a form of voice show.

A transcranial Doppler-based system for displaying three-dimensional intracranial blood flow information, wherein, the system includes: a probe scanning module, applied to generating an ultrasound and scanning the predetermined intracranial areas with a multi-beam ultrasound; an ultrasound receiving module, applied to receiving the ultrasonic echo signal; a probe scanning control module, applied to controlling the ultrasonic probes; a data processing module, applied to calculating and achieving the blood flow information including the depth of the intracranial blood vessels, the blood flow direction, the relative blood flow volume and the blood flow velocity, according to the ultrasonic echo signals; a 3D image drawing module, applied to visualization processing the data of the stereoscopic model (to form the 3D images. and present to the users) and presenting to the users; and a 3D imaging parameters control module, applied to performing individualized adjustments to the 3D images before presenting to the users, according to the users' commands.

The system for displaying three-dimensional intracranial blood flow information, wherein, the system further includes a parameter measurement module, applied to calculating the diameter of the blood vessel by slicing the 3D model; and calculating the blood flow volume information of the blood vessel according to the diameter of the blood vessel and the blood flow velocity. The system for displaying three-dimensional intracranial blood flow information, wherein, the data processing module is further applied to filtering out a plurality of non-blood flow information in the ultrasonic echo signals before running a 3D modeling calculation; and according to a predetermined standard, processes an ultrasonic echo signal in a different depth by adopting a calculation method with a different calculation complexity and accuracy. The system for displaying three-dimensional intracranial blood flow information, wherein, the system further includes a voice output module, applied to outputting the blood flow information to the sound card, and presenting to the users in a form of voice show.

Benefits: the present invention provides a transcranial Doppler-based method and system for displaying three-dimensional intracranial blood flow information, by scanning a plurality of intracranial areas with an ultrasound, obtaining the blood flow information (velocity, blood flow volume, direction) of each depth in all scanning directions, and further obtaining the 3D distribution figure of the blood flow by a data reconstruction. Comparing to the MRI, CT, DSA or other methods, the present invention is low cost, convenient and may be applied for repeated examination, it is able to make a long term monitoring of the global intracranial blood flow status or the blood flow information of one or a plurality of areas with the predetermined features. Comparing to the transcranial Doppler imaging or the transcranial color Doppler imaging in the prior art, the present invention may significantly reduce a degree of reliance on an operator's experience, and may further be able to provide a more complete and objective blood flow information, to facilitate a doctor to diagnose.

### BRIEF DESCRIPTION OF THE DRAWINGS

FIG. 1 illustrates a functional block diagram of a TCD device applied to achieving the method for displaying the three-dimensional intracranial blood flow information as provided in an embodiment of the present invention;

FIG. 2 illustrates a flow chart of the transcranial Doppler-based method for displaying the three-dimensional intracranial blood flow information as provided in an embodiment of the present invention;

FIG. 3 illustrates a schematic diagram of a probe scanning module adopting the mechanical control method as provided in an embodiment of the present invention.

### DETAILED DESCRIPTION OF EMBODIMENTS

The present invention provides a transcranial Doppler-based method and system for displaying three-dimensional intracranial blood flow information. In order to make the purpose, technical solution and the advantages of the present invention clearer and more explicit, further detailed descriptions of the present invention are stated here, referencing to the attached drawings and some preferred embodiments of the present invention. It should be understood that the detailed embodiments of the invention described here are used to explain the present invention only, instead of limiting the present invention.

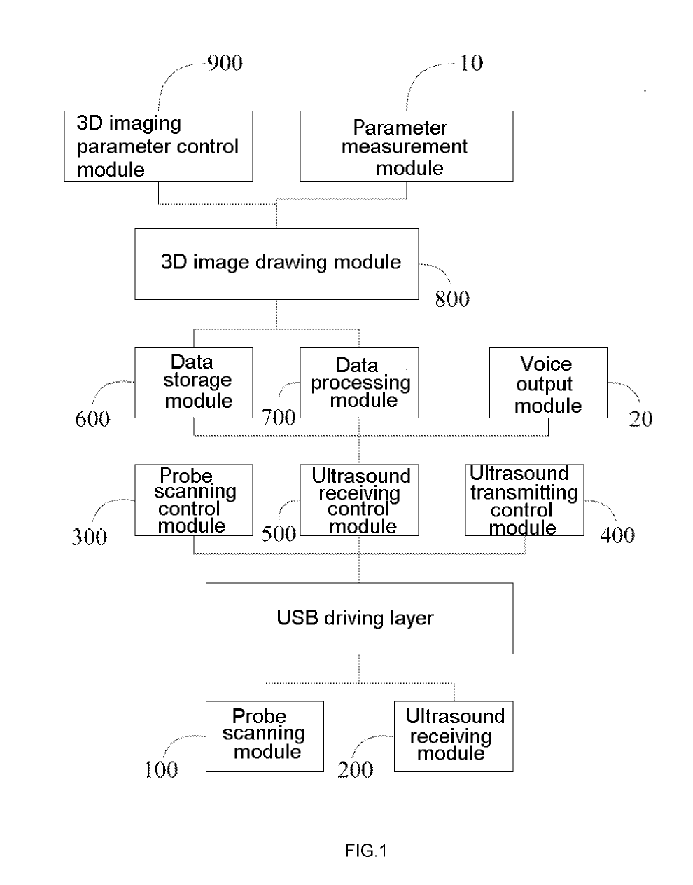

Referencing to FIG. 1, which is a functional block diagram of a TCD device applied to achieving the method for displaying the three-dimensional intracranial blood flow information as provided in an embodiment of the present invention. The TCD device includes a probe scanning module 100, applied to sending an ultrasound and scanning a plurality of areas, an ultrasound receiving module 200, a probe scanning control module 300, an ultrasound transmitting control module 400, an ultrasound receiving control module 500, a data storage module 600, a data processing module 700, a 3D image drawing module 800 and a 3D imaging parameter control module 900.

Specifically, the probe scanning module 100 may achieve a scanning the predetermined intracranial areas with a multi-beam ultrasound by using a mechanical control method of controlling a deflection of the ultrasound probe or using an electron-focusing method of an ultrasonic multi-array element probe, or using a method of combining the mechanical control method and the electron-focusing method.

In an embodiment of the present invention, scanning a plurality of blood vessels in a specific intracranial area is achieved by controlling a deflection angle of the ultrasound probe with two (or more) stepper motors. Of course, it may also choose other suitable methods to achieve a mechanical control of the probe.

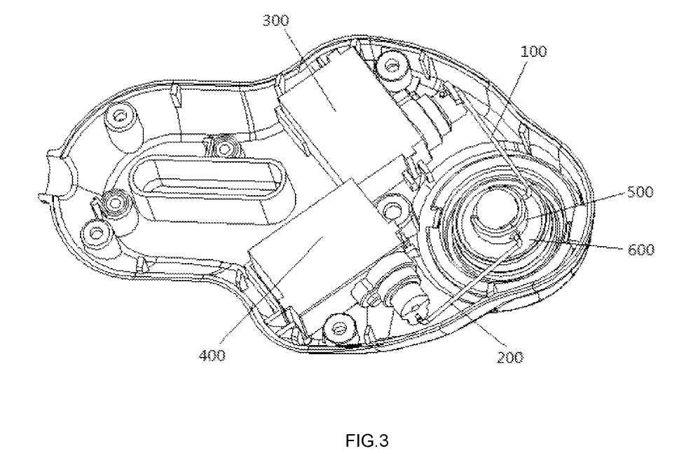

Shown as FIG. 3, specifically, first, connecting one end of each of a connection rod 100 and a connection rod 200 to a stepper motor 300 and a stepper motor 400 respectively. Another end of each of the two connection rods connects each other at a connection point and the two connection rods are kept perpendicular to each other in a same plane.

Followed by connecting the connection point of the two connection rods and one end of an ultrasound probe vibration element 500 to an end point. An end of the vibration element is fixed by an elastic material 600, while keeping the ultrasound probe perpendicular to both of the connection rods.

Since the ultrasound probe vibration element 500 is fixed by an elastic material 600, thus by controlling a rotation of the stepper motor to control a motion of the connection rods may make the probe deflect within a predetermined range of angle, so as to achieve a scanning to the blood vessels in a certain area.

The ultrasound receiving module 200 is applied to receiving an ultrasonic echo signal, the probe scanning control module 300 and the ultrasound transmitting control module 400 are applied respectively to controlling a mechanic movement of the probe (such as controlling the stepper motor to control the deflection angle of the probe) and a transmitting control logic of the ultrasound, which controls a plurality of parameters including a transmitting power, a transmitting frequency and more.

The ultrasound receiving control module 500 is applied to achieving receiving the ultrasonic echo signals or data. The data storage module 600 is applied to storing a plurality of related data and programs, including a plurality of data on the ultrasound echo, the 3D imaging and more.

The data processing module 700 is applied to making a 3D modeling and calculation to the intracranial blood flow through a plurality of ultrasonic echo signals in multi-depth and multi-angle. Since it is possible to obtain a plurality of information including a velocity of the blood flow, a direction, a depth and a relative blood flow volume from the ultrasound probe echo signals, thus, after the ultrasound probe has made a regional scanning controlled by the stepper motor, it is possible to achieve a stereoscopic model including the blood flow velocity, direction, depth and relative blood flow volume. And, the data processing module 700 may further slice the stereoscopic model to calculate the diameter of the blood vessel. Preferably, before the 3D modeling and calculation, the data processing module 700 may further filter out a plurality of non-blood flow information in the ultrasonic echo signals; and according to a predetermined standard, adopting a calculation method with a different calculation complexity and accuracy for an ultrasonic echo signal in a different depth. The data processing module 700 works by using any suitable computer platform owning a certain calculation abilities.

In a preferred embodiment of the present invention, the system may further include a parameter measurement module 10, applied to calculating the blood flow volume information of the blood vessel according to the diameter of the blood vessel and the blood flow velocity.

The 3D image drawing module 800 is applied to visualization processing the data of the stereoscopic model before forming the 3D images and presenting to the users. The 3D imaging parameter control module 900 is applied to performing individualized adjustments to the 3D images before presenting to the user according to a user commands, such as changing a scope of analysis, chromatography, and displaying a plurality of according blood flow information data and more.

More preferably, the system may further include a voice output module 20, applied to outputting the blood flow information to a sound card, and presenting to the user in a form of voice. Through the above said methods, it is possible to facilitate a doctor to observe and understand a situation of the blood flow from an angle of auditory.

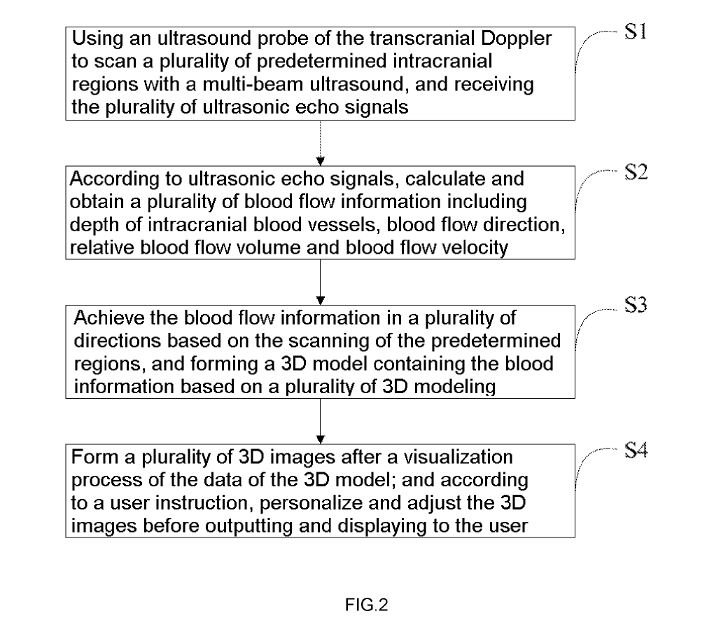

Shown as FIG. 2, which is a transcranial Doppler-based method for displaying the three-dimensional intracranial blood flow information as provided in an embodiment of the present invention. The method includes a plurality of following steps:

S1. Using an ultrasound probe of the transcranial Doppler to scan a plurality of predetermined intracranial areas with a multi-beam ultrasound, and receiving the plurality of ultrasonic echo signals.

S2. According to the ultrasonic echo signals, calculate and obtain a plurality of blood flow information including the depth of a plurality of intracranial blood vessels, the blood flow direction, the relative blood flow volume and the blood flow velocity.

A principle of the above said calculations is specifically:

1. The direction, velocity and energy: using a gate circuit to make the ultrasound probe generate a pulse ultrasound, according to a principle of Doppler frequency shift, when an ultrasound touches a red blood cell in an intracranial blood vessel, it will make a frequency shift, through receiving a plurality of related data on the ultrasonic echo, we may obtain the direction, velocity and energy of the blood flow. A specific calculation method may use a fast Fourier transform, a complex correlation operation or a Doppler energy calculation and more.

2. Depth: as mentioned above, due to adopting an ultrasound in a pulsed emission, given a known time interval and a known ultrasound speed, a distance between the blood vessel and the ultrasound probe (i.e., the depth) may be calculated from the time when the echo is received,

S3. Achieve the blood flow information in a plurality of directions based on the scanning of the predetermined areas, and forming a stereoscopic model containing the blood information based on a plurality of 3D modeling calculations. Since it is a full range ultrasound scanning to the predetermined area, thus it is possible to obtain a plurality of data in a plurality of directions in the area (i.e., the echo information of the multi-beam ultrasound). A stereoscopic model may be achieved through integrating these data by a commonly used 3D modeling calculation.

Of course, the predetermined areas are decided by a real case, such as a position of an intracranial blood vessel that a doctor needs to monitor. The predetermined areas may also be a plurality of small areas, or a plurality of small areas divided from a certain large predetermined area, which is ultrasonic scanned individually.

After achieving a plurality of 3D images of predetermined areas, integrates the images and obtains more complete intracranial blood vessels information. For example, it may scan both left and right temporal windows respectively to obtain two images of 3D blood distribution, then through compositing the images, a more accurate cerebral blood flow 3D diagram will be obtained. S4. Form a plurality of 3D images after a visualization process of the data of the stereoscopic model; and according to a user commands, personalize and adjust the 3D images before outputting and presenting to the user. In a real display, it is further possible to mark and display a plurality of blood flow information at a certain point including the velocity of the blood flow, the direction, the energy and more, in the 3D images.

Specifically, as described above, in an embodiment of the present invention, it is possible to obtain the multi-beam ultrasound for scanning the predetermined intracranial areas by using a mechanical control method of a deflection of the ultrasonic probe or using an electron-focusing method of an ultrasonic multi-array element probe, or using a method of combining the mechanical control method and the electron-focusing method.

Preferably, the method further includes: slicing the stereoscopic model to calculate a diameter of the blood vessel; and calculate blood flow volume information of the blood vessel according to the diameter of the blood vessel and the velocity of the blood flow. Both the above said information on the diameter of the blood vessel and the blood flow volume may be marked in the corresponding blood flow area in the 3D images. After obtaining the information on the blood flow volume, it is possible to analysis symmetry between a left side arterial blood flow volume and a right side arterial blood flow volume, for a further diagnose.

In an embodiment of the present invention, the method may further include: according to the 3D images, make a long term monitoring on the blood flow information of one or a plurality of areas having a plurality of predetermined features. The said predetermined features are determined specifically by any real situations and requirements, such as a maximum blood flow velocity area, a most narrow blood vessel area, a maximum blood flow changing area. The above said long term monitoring to a certain area may facilitate a doctor a lot for a treatment and diagnose.

Filtering out a plurality of non-blood flow information facilitates the data processing module generate the 3D images faster, reducing a pressure of data processing. The calculation method with different calculation complexity and accuracy means that a calculation method with a higher computational complexity is generally more accurate, while a calculation method with a lower computational complexity is often less accurate. Due to a fact that a complex calculation, such as a fast Fourier Transform (FFT), is not always required to all ultrasound echo signal calculations, therefore, based on the actual situation, the appropriate use of some simple calculations for echo signals may greatly reduce the overall amount of calculations.

More specifically, the method may further include: outputting the blood flow information to a sound card, and presenting to the user in a form of voice. After outputting to the sound card, connecting to a corresponding audio device may broadcast the blood flow information in a form of voice.

### Embodiment 1

A method of achieving the intracranial blood vessel 3D images construction using a transcranial Doppler ultrasound apparatus having an automatic monitoring detector from Delica medical equipment company:

First, make an intracranial scan from the left temporal window, a scan line number of the probe is 31*31=961, an angle difference between every two lines is about 1.47°, sampling points in each scan line are 128 points, an interval between two points is 1 mm.

Next, make a multiple correlation calculation to the ultrasound echo of each line, and obtain 128 blood flow energies and directions information. In such a way, from one side of the temporal window, it may obtain the data of 123008 accumulate intracranial points, and after the 3D reconstruction, an intracranial blood flow 3D diagram is obtained.

Afterwards, control the automatic probe rescan a plurality of beam positions with blood flow information displaying, and make the fast Fourier transform to the echo, before obtaining the blood flow parameter information of each point.

After finishing the left side scanning, make an intracranial scanning from the right temporal window, and obtain a 3D diagram on the right side intracranial blood flow and the blood flow parameter information of each point.

Finally, superimpose the 3D blood flow diagrams obtained from both left side and right side, and generate a more complete intracranial 3D diagram.

In a real application, it is possible to calculate an area of each major arterial blood vessel according to a thickness of different blood flow segments shown in the diagram, together with the flow velocity measured of each blood vessel, the blood flow volumes of different blood vessels may be obtained for different control and analysis.

It should be understood that, the application of the present invention is not limited to the above examples listed. Ordinary technical personnel in this field can improve or change the applications according to the above descriptions, all of these improvements and transforms should belong to the scope of protection in the appended claims of the present invention.

What is claimed is:

1. A transcranial Doppler-based method for displaying three-dimensional intracranial blood flow information, wherein, the method includes:
A. using an ultrasonic probe of the transcranial Doppler to scan a plurality of predetermined intracranial areas with a multi-beam ultrasound, and receiving a plurality of ultrasonic echo signals;
B. according to the ultrasonic echo signals, calculating and achieving a plurality of blood flow information including a depth of a plurality of intracranial blood vessels, a blood flow direction, a relative blood flow volume and a blood flow velocity;
C. achieving the blood flow information in a plurality of directions based on the scanning of the predetermined areas, and forming a stereoscopic model containing the blood flow information based on a plurality of 3D modeling calculations;
D. forming a plurality of 3D images after visualizing the data of the stereoscopic model; and according to a user command, performing individualized adjustments to the 3D images, and outputting the image in order to present to the user.

2. The method for displaying the three-dimensional intracranial blood flow information according to claim 1, wherein, the multi-beam ultrasound for scanning the predetermined intracranial areas is obtained by using a mechanical control method of a deflection of the ultrasonic probe or using an electron-focusing method of an ultrasonic multi-array element probe, or using a method of combining the mechanical control method and the electron-focusing method.

3. The method for displaying the three-dimensional intracranial blood flow information according to claim 1, wherein, the method further includes: E. slice the stereoscopic model to calculate a diameter of the blood vessel; and calculate blood flow volume information of the blood vessel according to the diameter of the blood vessel and the velocity of the blood flow.

4. The method for displaying the three-dimensional intracranial blood flow information according to claim 1, wherein, the method further includes: according to the 3D images, make a long term monitoring on an intracranial blood flow status in a whole, or on the blood flow information of one or a plurality of areas having a plurality of predetermined features.

5. The method for displaying the three-dimensional intracranial blood flow information according to claim 1, wherein, the step B further includes: before running the 3D modeling calculation, filter out a plurality of non-blood flow information in the ultrasonic echo signals; and according to a predetermined standard, adopt a calculation method with a different calculation complexity and accuracy for the ultrasonic echo signals in a different depth.

6. The method for displaying the three-dimensional intracranial blood flow information according to claim 1, wherein, the method further includes: output the blood flow information to a sound card, and present to the user in a form of voice show.

7. A transcranial Doppler-based system for displaying a three-dimensional intracranial blood flow information, wherein, the system includes: a probe scanning module, applied to generating an ultrasound and scanning a predetermined intracranial areas with a multi-beam ultrasound; an ultrasound receiving module, applied to receiving an ultrasonic echo signal; a probe scanning control module, applied to controlling an ultrasound probes; a data processing module, applied to calculating and achieving the blood flow information including a depth of an intracranial blood vessels, a blood flow direction, a relative blood flow volume and a blood flow velocity, according to the ultrasonic echo signals; a 3D image drawing module, applied to visualization processing a data of a stereoscopic model to form the 3D images, and presenting to users; and a 3D imaging parameters control module, applied to performing individualized adjustments to a 3D images before presenting to the users, according to the user commands.

8. The system for displaying the three-dimensional intracranial blood flow information according to claim 7, wherein the system further includes a parameter measurement module, applied to calculating the diameter of the blood vessel by slicing the stereoscopic model; and calculating the blood flow volume information of the blood vessel according to the diameter of the blood vessel and the blood flow velocity.

9. The system for displaying the three-dimensional intracranial blood flow information according to claim 7, wherein the data processing module is further applied to, before running a 3D modeling calculation, filtering out a plurality of non-blood flow information in the ultrasonic echo signals; and according to a predetermined standard, adopt a calculation method with a different calculation complexity and accuracy for the ultrasonic echo signal in a different depth.

10. The system for displaying the three-dimensional intracranial blood flow information according to claim 7, wherein, the system further includes a voice output module, applied to outputting the blood flow information to a sound card, and presenting to the user in a form of voice show.
